(19) United States
(12) Patent Application Publication
(10) Pub. No.: US 2024/0215948 A1
(43) Pub. Date: Jul. 4, 2024

(54) MEDICAL SCANNING DEVICE

(71) Applicant: GE Precision Healthcare LLC, Waukesha, WI (US)

(72) Inventors: Minyu Gu, Wuxi (CN); Zhiwen Wang, Wuxi (CN); Ke Tao, Wuxi (CN); Gang Liu, Wuxi (CN); Feng Wu, Wuxi (CN); Weijie Zhang, Wuxi (CN)

(21) Appl. No.: 18/541,916

(22) Filed: Dec. 15, 2023

(30) Foreign Application Priority Data
Dec. 30, 2022 (CN) 202211726311.7

(51) Int. Cl. A61B 8/00; A61B 6/46; G16H 10/65; G16H 30/20

(52) U.S. Cl. CPC A61B 8/4245; A61B 6/462; A61B 8/4461; G16H 10/65; G16H 30/20; A61B 2560/02

(57) ABSTRACT

A medical scanning device used to scan a subject to be scanned, and obtain a medical image obtained by a current scan, and current scan-related information; and store the scan-related information in an identifiable medium, the identifiable medium being provided in a portable mobile carrier. The scan-related information is saved in the identifiable medium, and the identifiable medium is provided in the portable mobile carrier, so that the portable mobile carrier can be used to easily implement offline transfer of the scan-related information, and doctors can quickly and conveniently acquire the scan-related information so as to find a lesion or an issue of concern, thereby making medical scanning across devices, hospitals, cities, and countries simple and reducing costs.

### CROSS-REFERENCE TO RELATED APPLICATION

The present application claims priority to Chinese patent application Ser. No. 20/221,1726311.7, filed on Dec. 30, 2022. The entire contents of the above-listed application are incorporated by reference herein in their entirety.

### TECHNICAL FIELD

Embodiments of the present application relate to the technical field of medical devices, and relate in particular to a medical scanning device and associated method.

### BACKGROUND

Some patients need to regularly undergo medical scans of body parts. Before each scan, a doctor needs to read previous scan reports provided by a patient, or retrieve scan records for the patient locally saved by a current scanning device, or connect to a network to retrieve scan records for the patient saved on a server, so as to acquire more scan guidance information.

It should be noted that the above introduction of the background is only for the convenience of clearly and completely describing the technical solutions of the present application, and for the convenience of understanding for those skilled in the art.

### SUMMARY

Embodiments of the present application provide a medical scanning method and a medical device.

According to an aspect of the embodiments of the present application, a medical scanning method is provided. The medical scanning method comprises:
scanning a subject to be scanned, and obtaining a medical image obtained by a current scan, and current scan-related information; and
storing the scan-related information in an identifiable medium, the identifiable medium being provided in a portable mobile carrier.

According to an aspect of the embodiments of the present application, a medical device is provided. The medical device comprises:
a scanning probe, the scanning probe scanning a subject to be scanned; and
a processor, the processor obtaining a medical image obtained by a current scan, and current scan-related information, and storing the scan-related information in an identifiable medium, the identifiable medium being provided in a portable mobile carrier.

One of the benefits of the embodiments of the present application is as follows: the scan-related information is saved in the identifiable medium, and the identifiable medium is provided in the portable mobile carrier, so that the portable mobile carrier can be used to easily implement offline transfer of the scan-related information, and doctors can quickly and conveniently acquire the scan-related information so as to find a lesion or an issue of concern, thereby making medical scanning across devices, hospitals, cities, and countries simple and reducing costs.

With reference to the following description and drawings, specific implementations of the embodiments of the present application are disclosed in detail, and the means by which the principles of the embodiments of the present application can be employed are illustrated. It should be understood that the implementations of the present application are therefore not limited in scope. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive skill. In the drawings:

FIG. 1 is a schematic diagram of a medical scanning method according to an embodiment of the present application;

FIGS. 2A and 2B are schematic diagrams of a thyroid visual representation image and a breast visual representation image according to an embodiment of the present application;

FIG. 3 is a schematic diagram of a medical scanning method according to an embodiment of the present application;

FIGS. 4A and 4B are schematic diagrams of a thyroid body marker image and a breast body marker image according to an embodiment of the present application;

FIG. 5 is a schematic diagram of a graphical user interface according to an embodiment of the present application;

FIG. 6 is a schematic diagram of a control device according to an embodiment of the present application;

FIG. 7 is a schematic diagram of a medical device according to an embodiment of the present application;

FIG. 8 is a schematic diagram of an ultrasonic device according to an embodiment of the present application;

FIG. 9 is a schematic diagram of a medical scanning method according to an embodiment of the present application; and

FIG. 10 is a schematic diagram of a scanning probe icon according to an embodiment of the present application.

### DETAILED DESCRIPTION

The foregoing and other features of the embodiments of the present application will become apparent from the following description and with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have” etc., refer to the presence of described features, elements, components, or assemblies, but do not exclude the presence or addition of one or more other features, elements, components, or assemblies.

In the embodiments of the present application, the singular forms “a” and “the”, etc. include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

Currently, when a patient regularly undergoes scans of a body part, there are the following scenarios:
1) the patient may undergo scans by going to different hospitals, and/or using different scanning devices, and/or by doctors having different experience; and
2) the patient may perform scans by himself/herself using a home medical device.

In the above scenarios, there may be the following problems:
on the basis of restrictions in certain regions or hospital policies, the medical devices can only work within a local area network or can only work in an offline state. Therefore, when devices or doctors or hospitals are changed to perform body scans, the doctors cannot locally retrieve the patient's historical scan records, nor can the doctors retrieve the patient's historical scan records by connecting to a network. Therefore, scan guidance information cannot be easily acquired. The doctors, at each scan, may need to spend time reading the patient's scan reports, and may need to spend time adjusting scan parameters and the position of a scanning probe to find lesions or issues of concern. Even so, for less experienced doctors or patients, it is difficult to ensure alignment with previous scan parameters. Therefore, it is difficult to obtain scan images having similar scan quality.

In view of at least one of the above technical problems, embodiments of the present application provide a medical scanning method and a medical device.

The following is a specific description of an embodiment of the present invention with reference to the accompanying drawings.

The embodiments of the present application provide a medical scanning method, which is applied to a medical device.

In some embodiments, the medical device includes at least one of an electronic computed tomography device, a nuclear magnetic resonance device, an electron emission tomography device, a single photon emission computed tomography device, and an ultrasound device, but the present application is not limited thereto. The medical device may also be other devices that can acquire medical images.

FIG. 1 is a schematic diagram of a medical scanning method according to an embodiment of the present application. As shown in FIG. 1, the method includes:
101, scanning a subject to be scanned, and obtaining a medical image obtained by a current scan, and current scan-related information; and
102, storing the scan-related information in an identifiable medium, the identifiable medium being provided in a portable mobile carrier.

In some embodiments, in 101, a region of interest to be scanned (such as the thyroid, breast, etc.) of the subject may be determined, and preset scan parameters corresponding to the region of interest may be determined. The preset scan parameters may also be adjusted as needed. A scanning probe of a medical device applies a physical signal such as visible light, an X-ray, ultrasound, or a magnetic field to the region of interest of the subject to be scanned by adjusting a position and a pose, so as to scan the subject to be scanned and acquire a signal intensity distribution fed back signal by the subject to be scanned. A medical image obtained by a current scan is obtained by means of image reconstruction technology. The medical image can be a two-dimensional image, a three-dimensional image, or a four-dimensional image. Taking ultrasound imaging as an example, real-time non-invasive high-frequency sound waves are emitted toward the subject to be scanned by means of the scanning probe, so that reflected imaging data is acquired, and a corresponding medical image is generated in real time. The medical image acquired by the current scan refers to a medical image (anatomical image of a specific section) that can reflect the state (morphology) of an organ or tissue of the subject at a certain time.

In some embodiments, after obtaining the medical image, an operation such as measurement and diagnosis may be performed on the basis of the medical image to obtain scan-related information of the present scan. The scan-related information includes at least one of scan parameter information, body marker information, and scanning result information.

In some embodiments, the scan parameter information includes at least one of a scanning mode (e.g., mode B, mode C, mode M, 3D/4D mode, etc.), a scanning presetting (or a scanning protocol, such as a thyroid presetting or breast presetting, depth presetting, gain presetting, etc.), a scan parameter adjustment, and a scanning time. Some of the above scan parameters may be used to adjust image quality. The medical device may record the above scan parameter information (preset or adjusted) used when acquiring the medical image.

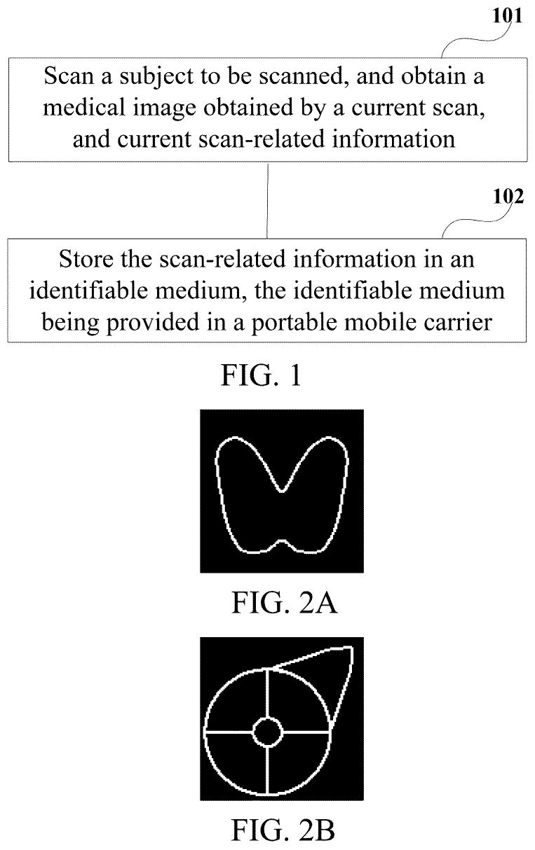

In some embodiments, the body marker information includes body marker type information and position and pose information of a scanning probe icon, and the position and pose information includes at least one of position information of the scanning probe icon, and angle of rotation information of the scanning probe icon, wherein corresponding visual representation images may be preset for different body parts or organs, and each visual representation image may be regarded as a schematic diagram of the corresponding body part or organ. FIGS. 2A and 2B are schematic diagrams of a breast visual representation image and a thyroid visual representation image according to an embodiment of the present application. The body marker type information may be represented using a body marker index, which is used to uniquely identify the corresponding visual representation image. For example, the body marker index of the thyroid visual representation image is 1, and the body marker index of the breast visual representation image is 2. The position information of the scanning probe icon reflects the location at which the scanning probe is placed at the corresponding body part or organ when the medical image is acquired, and the angle of rotation information of the scanning probe icon reflects the actual angle of rotation of the scanning probe when the medical image is acquired. The body marker information may be used to reconstruct a body marker image, thereby guiding a doctor to find a suitable scanning position on the body part or organ. Regarding how to reconstruct the body marker image, same will be described later.

For example, the position and pose information is information of the position of the scanning probe icon and/or the angle of rotation of the scanning probe icon in a visual representation image coordinate system, wherein the scanning probe icon is a rectangular frame including line segments and dots. That is, using the visual representation image coordinate system as a reference system, the coordinate position of a pixel point in the visual representation image is used as the position information of the scanning probe icon, which may also be regarded as center position information of the rectangular frame, and an angle, in the visual representation image, relative to an X-axis or Y-axis of the visual representation image coordinate system is used to represent the angle of rotation of the scanning probe icon, which may also be regarded as the included angle between one side of the rectangular frame and the X-axis or Y-axis. The foregoing is only an illustrative description, and the embodiments of the present application are not limited thereto. FIG. 10 is a schematic diagram of the scanning probe icon according to an embodiment of the present application. As shown in FIG. 10, the rectangular frame represents the range of the scanning probe icon, and a line segment 1001 (line segment length) in the rectangular frame symbolizes the coverage range of the scanning probe, or is the range of contact between the scanning probe (e.g., an ultrasound probe) and the region of interest of the subject to be scanned. A dot 1002 represents a starting position of an edge of the coverage range of the scanning probe, or represents the orientation of the scanning probe.

In some embodiments, the scanning result information includes at least one of a measurement result and diagnostic information. For example, the medical image includes a lesion, the measurement result includes at least one of the width, height, length, area, and volume of the lesion, and the diagnostic information includes the grade of the lesion. The foregoing is only an example, and the embodiments of the present application are not limited thereto. For example, the medical image includes a fetal image, the measurement result includes at least one of the head circumference, body length, and abdominal circumference of a fetus, and the diagnostic information includes a fetal abnormality result, etc. No more examples will not be given here.

In some embodiments, in 102, the scan-related information is stored in an identifiable medium. The identifiable medium may be at least one of a barcode, a two-dimensional code, a three-dimensional code, a near field communication (NFC) tag, and a radio frequency identification (RFID) tag. That is, the above scan-related information may be saved in the identifiable medium by means of corresponding encoding, encryption, and compression technology. For example, the above scan-related information may be represented using a character string, and the scan-related information is saved in the identifiable medium by means of encoding, encryption, and compression technology for the character string.

For example, when the identifiable medium is the two-dimensional code, the two-dimensional code includes: a PDF417 barcode, a Code 49 barcode, a Code 16K barcode, a Code One barcode, a Data Matrix barcode or a QR barcode, and the character string of the scan-related information is encoded according to a corresponding two-dimensional code format, encrypted (e.g., by an AES or RSA encryption algorithm) and data-compressed (e.g., by a zlib-deflate compression algorithm) to generate a two-dimensional code saving the scan-related information.

For example, when the identifiable medium is an NFC tag, the character string of the scan-related information is converted into ciphertext according to a predetermined format, and the ciphertext is written into a secret key region of the NFC tag to generate an NFC tag saving the scan-related information.

The above is described using the two-dimensional code and the NFC tag, respectively, as examples. For implementation methods of storing the scan-related information on other identifiable media, reference may be made to related technologies, and the embodiments of the present application are not limited thereto.

In some embodiments, the identifiable medium storing the scan-related information is provided in a portable mobile carrier, and the portable mobile carrier includes an electronic mobile terminal, or a printable medium, or an integrated circuit card, or the like. For example, the electronic mobile terminal may be a mobile phone, a tablet, a smart watch, or the like. When the identifiable medium is a barcode, a two-dimensional code, or a three-dimensional code, the identifiable medium storing the scan-related information may be sent to the electronic mobile terminal, or the electronic mobile terminal may be an electronic mobile terminal having an NFC function or an RFID function, and the identifiable medium may be an NFC tag or an RFID tag built in the electronic mobile terminal. For example, the electronic mobile terminal may be a printable medium, including: a paper examination report, a case, etc., and when the identifiable medium is a barcode, a two-dimensional code, or a three-dimensional code, the identifiable medium storing the scan-related information may be printed on the printable medium. For example, the electronic mobile terminal may be an integrated circuit card having an NFC function or an RFID function, and the identifiable medium may be an NFC tag or an RFID tag built in the integrated circuit card.

Therefore, compared with saving the scan-related information locally or in a cloud or on a server, in the embodiments of the present application, the portable mobile carrier can be used to easily implement offline transfer of the scan-related information carried by the identifiable medium, and doctors can quickly and conveniently acquire the scan-related information so as to find a lesion or an issue of concern, thereby making medical scanning across devices, hospitals, cities, and countries simple and reducing costs.

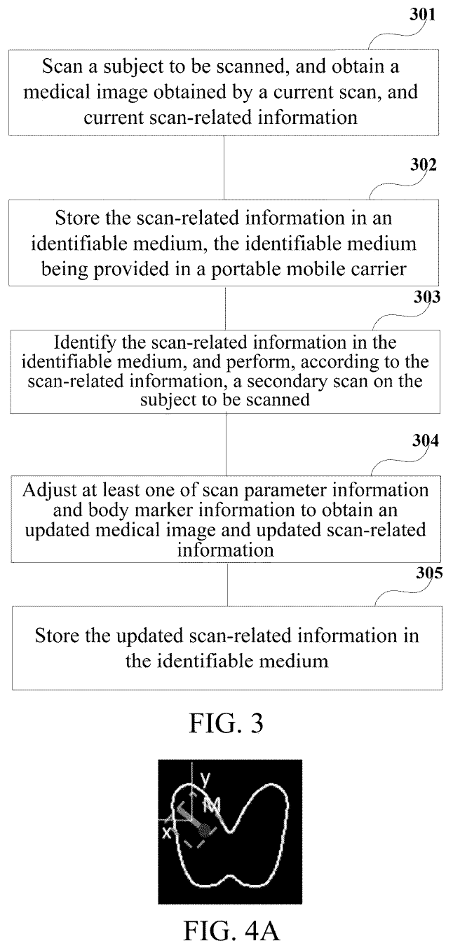

FIG. 3 is a schematic diagram of a medical scanning method according to an embodiment of the present application. As shown in FIG. 3, the method includes:
301, scanning a subject to be scanned, and obtaining a medical image obtained by a current scan, and current scan-related information;
302, storing the scan-related information in an identifiable medium, the identifiable medium being provided in a portable mobile carrier; and
303, identifying the scan-related information in the identifiable medium, and performing a secondary scan on the subject to be scanned according to the scan-related information.

Regarding the embodiments of 301-302, reference may be made to 101-102, and the repeated parts will not be described again.

In some embodiments, the secondary scan performed in 303 and the scan performed in 301 (referred to hereinafter as a previous scan) may be scans performed by the same or different medical devices, and the present application is not limited thereto.

In some embodiments, the medical device may include an identification apparatus for identifying the scan-related information in the identifiable medium. For example, the identification apparatus may be a barcode reader, or a two-dimensional code reader, or a three-dimensional code reader, or an NFC reader, or an RFID reader. When the identifiable medium is a barcode, or a two-dimensional code, or a three-dimensional code, the barcode reader, or the two-dimensional code reader, or the three-dimensional code reader scans the barcode, or the two-dimensional code, or the three-dimensional code on the portable mobile carrier, and identifies the scan-related information in the barcode, or the two-dimensional code, or the three-dimensional code by means of corresponding decoding, decryption, and decompression technology. When the identifiable medium is an NFC tag, the NFC reader can read the scan-related information in the NFC tag when the portable mobile carrier is in the NFC range of the NFC reader. When the identifiable medium is an RFID tag, the RFID reader can read the scan-related information in the RFID tag when the portable mobile carrier is in an RFID range near the RFID reader. Regarding the embodiments of reading the scan-related information by various readers, reference may be made to related technologies, and they will not be repeated here.

In some embodiments, after the scan-related information is identified, to conveniently and intuitively view the scan-related information for guidance on the secondary scan, the method may further include: displaying the scan-related information. The medical device is electrically connected to a display, or the medical device has a display. The display may be used to display a graphical user interface, and the graphical user interface may include a region for displaying the medical image acquired by the medical device. The scan-related information may be superimposed and displayed on the medical image obtained by the secondary scan. Taking ultrasound imaging as an example, when performing the secondary scan, a corresponding medical image may be generated on the display in real time, and the scan-related information is superimposed and displayed on the medical image.

For example, when the scan-related information is scan parameter information or scanning result information, a parameter name of the scan parameter information and a corresponding parameter value may be superimposed and displayed on the medical image (in a list mode). Alternatively, a result name of the scanning result information and a corresponding result value may be superimposed and displayed (in a list mode).

For example, when the scan-related information is body marker information, a body marker image may be generated according to the body marker information of the scan-related information, and the body marker image is superimposed and displayed on the medical image obtained by the secondary scan. The body marker image includes a visual representation image of a scanned site and a scanning probe icon superimposed and displayed on the visual representation image. It should be noted that in the foregoing embodiment, the character string of the body marker information being encoded, encrypted, and compressed to the identifiable medium is described as an example, and optionally, the body marker image may also be image-encoded, encrypted, and compressed and then stored into the identifiable medium. The embodiments of the present application are not limited thereto.

How to generate the body marker image will be further described below. First, a corresponding visual representation image is determined according to body marker type information in the body marker information. For example, if the body marker type information, i.e., the body marker index, is 1, then the visual representation image is determined to be a thyroid visual representation image. Then, the position and angle of rotation of the scanning probe icon in the visual representation image are determined according to position and pose information of the scanning probe icon in the body marker information. For example, the position of one pixel in the visual representation image is determined according to the position information of the scanning probe icon, the position of the pixel is used as the center position of the rectangular frame of the scanning probe icon, and the angle of rotation of the rectangular frame of the scanning probe icon is determined according to the angle of rotation of the scanning probe icon, thereby superimposing the scanning probe icon in the visual representation image. According to the body marker image, the doctor and the patient can intuitively know the angle of rotation and position of the scanning probe.

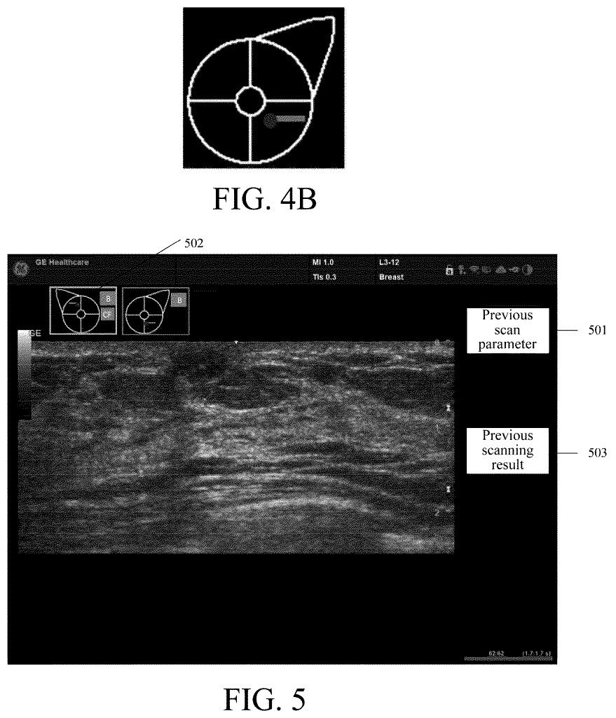

FIG. 4A is a schematic diagram of a thyroid body marker image generated according to an embodiment of the present application. As shown in FIG. 4A, the rectangular frame may represent the range of the scanning probe icon, the coordinates of the center position of the rectangular frame in the visual representation image coordinate system are equal to the position information of the scanning probe icon, and the included angle between one side M of the rectangular frame in the visual representation image coordinate system and the Y-axis is equal to the angle of rotation of the scanning probe icon. A line segment (line segment length) in the scanning probe icon symbolizes the coverage range of the scanning probe. The dot represents a starting position of an edge of the coverage range of the scanning probe. As shown in FIG. 4A, according to the body marker image, it is determined that the scanning probe is approximately located on an upper position of the right lobe of the thyroid. The dot is located at the lower right corner position of the line segment, indicating that a starting position of an edge of the scanning probe is toward the lower right corner. FIG. 4B is a schematic diagram of a breast body marker image generated according to an embodiment of the present application, wherein a generation means thereof is similar to that of FIG. 4A, and will not be repeated here.

FIG. 5 is a schematic diagram of a graphical user interface according to an embodiment of the present application. As shown in FIG. 5, a body marker image 501, scan parameter information 502, and scanning result information 503 are superimposed and displayed on a medical image.

In some embodiments, in 303, a secondary scan is performed, according to the scan-related information, on the subject to be scanned, including at least one of the following: setting, according to the scan parameter information, a scan parameter of the secondary scan, and determining, according to the body marker information, the position and pose of a scanning probe used in the secondary scan. Setting, according to the scan parameter information, the scan parameter of the secondary scan includes: setting the scan parameter of the secondary scan to be the same as scan parameter information of a previous scan. Determining, according to the body marker information, the position and pose of the scanning probe used in the secondary scan includes: setting the position and pose of the scanning probe used in the secondary scan to the same position and pose of the scanning probe of the previous scan. That is, when performing the secondary scan, reference may be made to the scan-related information of the previous scan as scan guidance information, so that the alignment with the historical scan parameters may be ensured, thereby enabling a scan image of similar scan quality to be obtained.

In some embodiments, optionally, the method may further include: 304, adjusting at least one of the scan parameter information and the body marker information to obtain an updated medical image and updated scan-related information.

For example, when performing the secondary scan, scan parameters of the present scan may be set (for example, automatically set by the system) to be the same as scan parameters of the previous scan. For example, the depth information, gain information, and scanning mode of the secondary scan are set to be the same as the depth information, gain information, and scanning mode in the identified scan parameter information. Optionally, all or part of the scan parameters of the secondary scan may also be adjusted. For example, the depth information is adjusted, and the other scan parameters remain aligned with the scan parameters of the previous scan. The embodiments of the present application are not limited thereto. The values of the adjusted scan parameter before and after adjustment may be superimposed and displayed on the medical image of the secondary scan, that is, in addition to superimposing and displaying the identified scan parameter (depth information) of the previous scan, the adjusted scan parameter (depth information) of the secondary scan may also be superimposed and displayed.

For example, when performing the secondary scan, the position and angle of rotation of the scanning probe of the present scan may be set to be the same (e.g., manually set) as the position and angle of rotation of the scanning probe of the previous scan. Optionally, the position or angle of rotation of the scanning probe of the secondary scan may also be adjusted. For example, the angle of rotation is adjusted, and the position remains unchanged. The embodiments of the present application are not limited thereto.

In some embodiments, for a method for obtaining the updated medical image and the updated scan-related information, reference may be made to 101. The doctor may perform an operation such as measurement and diagnosis on the basis of the updated medical image, and obtain scanning result information of the secondary scan, including at least one of an updated measurement result and updated diagnostic information.

In some embodiments, if the scan-related information of the secondary scan is updated, the method may further include: 305, storing the updated scan-related information in the identifiable medium. For example, when the identifiable medium is an NFC tag or an RFID tag, the updated scan-related information may be rewritten to the NFC tag or the RFID tag. When the identifiable medium is a barcode, or a two-dimensional code, or a three-dimensional code, a new barcode, or two-dimensional code, or three-dimensional code can be generated on the basis of the updated scan-related information. For example, part of the updated scan-related information of the secondary scan and the scan-related information of the previous scan are combined, encoded, encrypted, and compressed to generate a new barcode, or two-dimensional code, or three-dimensional code, which is then provided in a portable mobile carrier again. Thus, after the secondary scan, the identifiable medium has scan-related information of two scans stored therein, but the embodiments of the present application are not limited thereto. For example, only part of the updated scan-related information of the secondary scan may also be encoded, encrypted, and compressed to generate a new barcode, or two-dimensional code, or three-dimensional code. No more examples will be given here.

The above embodiment takes two scans as an example. When three or even more scans are further required, 303-305 may be performed repeatedly. No more examples will be given here. In addition, the scan-related information stored in the identifiable medium may include scan-related information of a total of N scans. For example, N is 3, which is merely an example here. The embodiments of the present application are not limited thereto. In addition, during each scan, the scan-related information superimposed and displayed on the medical image may be the scan-related information of the previous scan, and may also be the scan-related information of previous N scans, and the embodiments of the present application are not limited thereto.

The medical scanning method in the above embodiments may be applied to a scenario in which the medical device is offline or in a local area network, so that the portable mobile carrier can be used to easily implement offline transfer of the scan-related information, and doctors can quickly and conveniently acquire the scan-related information so as to find a lesion or an issue of concern, thereby making medical scanning across devices, hospitals, cities, and countries simple and reducing costs. However, the embodiments of the present application are not limited thereto, and the medical scanning method may also be applied to a scenario in which the medical device may be connected to a network.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

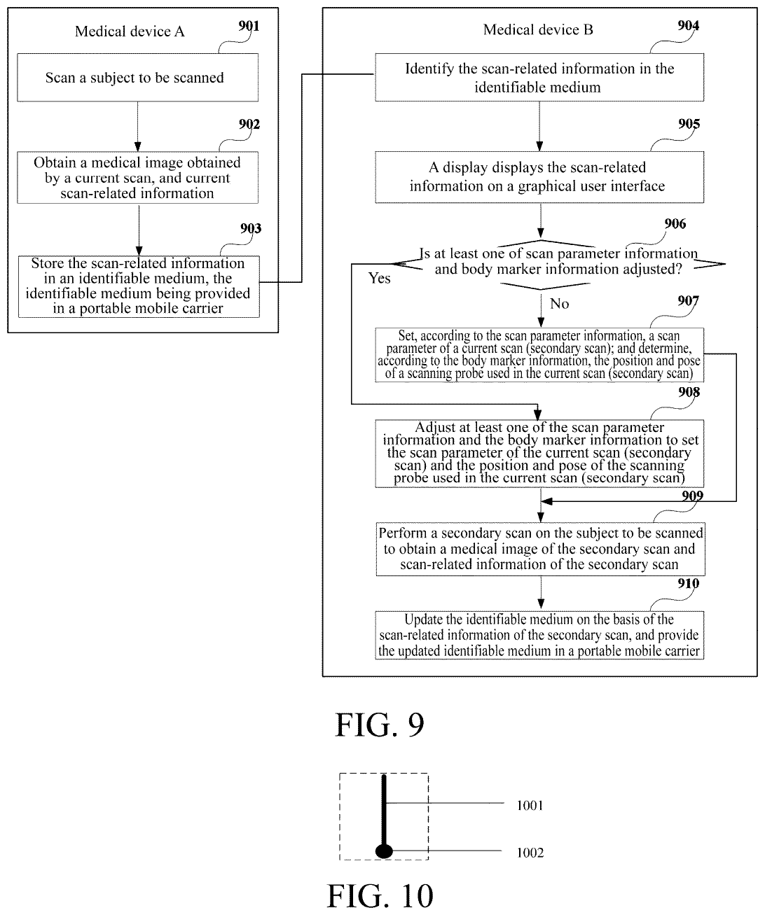

FIG. 9 is a schematic diagram of a medical scanning method according to an embodiment of the present application. As shown in FIG. 9, the method includes:
901, determining a region of interest (e.g., thyroid, breast, etc.) of the subject to be scanned, and recording a preset scan parameter corresponding to the region of interest, wherein the preset scan parameter may also be adjusted as needed, and a scanning probe of a medical device scans the subject to be scanned by adjusting the position and the pose;
902, obtaining a medical image obtained by a current scan and current scan-related information;
903, storing the scan-related information in an identifiable medium, the identifiable medium being provided in a portable mobile carrier, wherein, for example, the scan-related information is stored in a two-dimensional code, and the two-dimensional code is issued to an electronic mobile terminal of the subject to be scanned or the two-dimensional code is printed in an examination report of the subject to be scanned;
904, identifying the scan-related information in the identifiable medium;
905, displaying, by a display, the scan-related information on a graphical user interface, for example, including scan parameter information, body marker information, and scanning result information;
906, determining whether to adjust at least one of the scan parameter information and the body marker information, performing 908 when the adjustment is required, and performing 907 when the adjustment is not required;
907, setting, according to the scan parameter information, a scan parameter of the current scan (secondary scan), and determining, according to the body marker information, the position and pose of a scanning probe used in the current scan (secondary scan);
908, adjusting at least one of the scan parameter information and the body marker information to set the scan parameter of the current scan (secondary scan) and the position and pose of the scanning probe used in the current scan (secondary scan);
909, performing a secondary scan on the subject to be scanned to obtain a medical image of the secondary scan and scan-related information of the secondary scan; and
910, updating the identifiable medium on the basis of the scan-related information of the secondary scan, and providing the updated identifiable medium in a portable mobile carrier, wherein, for example, a two-dimensional code is re-generated, and the two-dimensional code is re-issued into the electronic mobile terminal of the subject to be scanned, or the new two-dimensional code is printed on the present examination report.

It should be noted that a medical device A executing 901 to 903 above and a medical device B executing 904 to 910 above may be the same medical device, and may also be different medical devices, and the embodiments of the present application are not limited thereto.

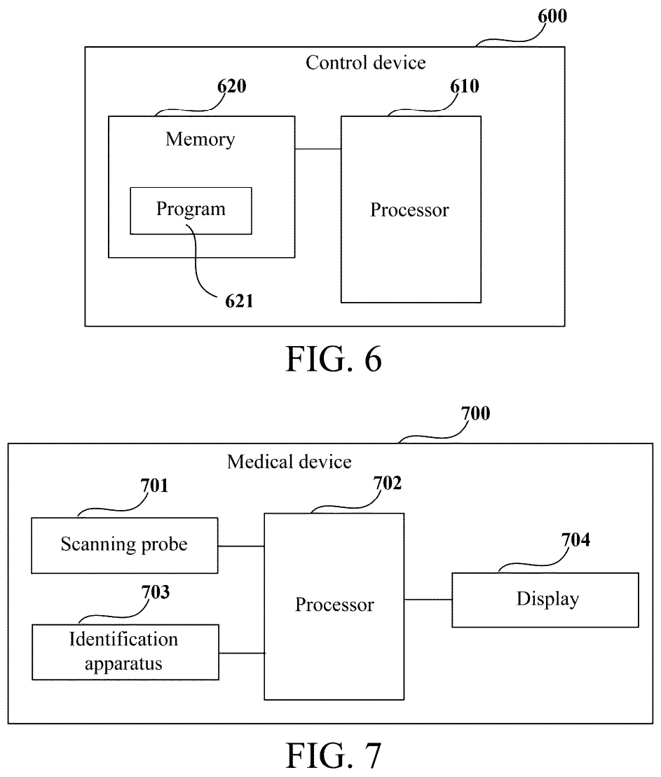

The embodiments of the present application further provide a control device. FIG. 6 is a schematic diagram of a control device according to an embodiment of the present application. As shown in FIG. 6, the control device 600 may include: one or more processors (for example, a central processing unit (CPU)) 610, and one or more memories 620. The memory 620 is coupled to the processor 610. The memory 620 can store various types of information, etc. In addition, the memory further inputs a control program 621 of a device, and the program 621 is executed under control of the processor 610. The memory 620 may include, for example, a ROM, a floppy disk, a hard disk, an optical disk, a magneto-optical disk, a CD-ROM, or a non-volatile memory card.

In some embodiments, the processor 610 is configured to control a scanning probe to scan a subject to be scanned, obtain a medical image obtained by a current scan, and current scan-related information, and store the scan-related information in an identifiable medium, the identifiable medium being provided in a portable mobile carrier. It may further include: setting, according to the scan parameter information, the scan parameter of the secondary scan. It may further include: storing the updated scan-related information in the identifiable medium. It may further include: generating a body marker image according to the body marker information of the scan-related information. For embodiments thereof, reference may be made to the foregoing embodiments, and they will not be repeated here.

It should be noted that the control device 600 does not necessarily include all of the components shown in FIG. 6. In addition, the control device 600 for the input device may further include components not shown in FIG. 6, for which reference may be made to related technologies.

The processor 610 may communicate with a medical device, a display, etc. The processor 610 may also be referred to as a microcontroller unit (MCU), a microprocessor, or a microcontroller, or other processor apparatuses and/or logic apparatuses. The processor 610 may include a reset circuit, a clock circuit, a chip, a microcontroller, etc. The functions of the processor 610 may be integrated on the motherboard of the medical device (e.g., the processor 610 is configured as a chip connected to a central processing unit (CPU) of the motherboard), or may be configured independently of the motherboard. The embodiments of the present application are not limited thereto.

For the sake of simplicity, FIG. 6 only exemplarily shows the connection relationship or signal direction between various components or modules, but it should be apparent to those skilled in the art that various related technologies such as a bus connection can be used. The various components or modules can be implemented by means of a hardware facility such as a processor or a memory, etc. The embodiments of the present application are not limited thereto.

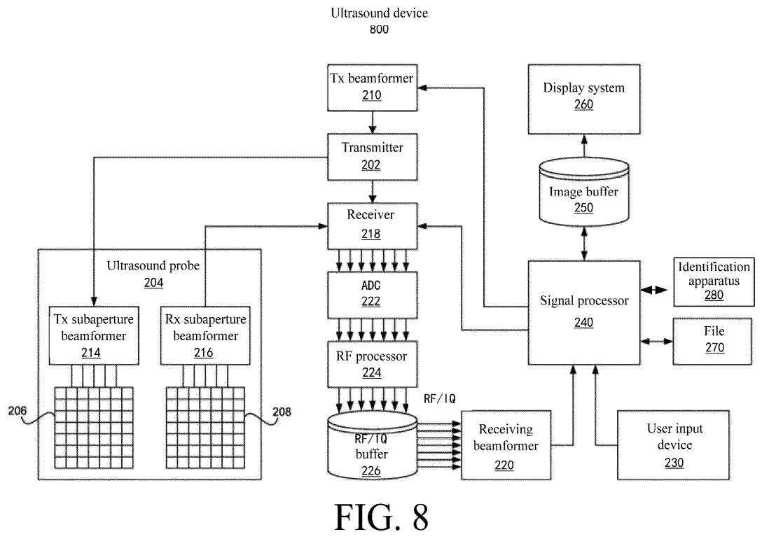

The embodiments of the present application further provide a medical device. FIG. 7 is a schematic diagram of a medical device according to an embodiment of the present application. For example, the medical device may be an ultrasound system or a magnetic resonance system configured to generate and/or render an ultrasound image, etc. FIG. 8 depicts an exemplary specific implementation of an ultrasound system that may correspond to the medical device, and a detailed description will be provided later. As shown in FIG. 7, the medical device 700 includes a scanning probe 701 that scans a subject to be scanned, and a processor 702 that obtains a medical image obtained by a current scan, and current scan-related information, and stores the scan-related information in an identifiable medium, the identifiable medium being provided in a portable mobile carrier.

The scanning probe 701 may be configured to generate and/or capture a specific type of imaging signal (and/or data corresponding thereto) by means of, for example, moving over the subject to be scanned (or a portion thereof), and may include suitable circuitry for performing and/or supporting such a function. For example, in the case where the medical is an ultrasound system, the scanning probe 701 is an ultrasound probe and may emit an ultrasound signal and capture an echo ultrasound image.

In some embodiments, reference may be made to the control device 600 for embodiments of the processor 702, which will not be repeated here.

In some embodiments, the medical device further includes: an identification apparatus 703 that identifies the scan-related information in the identifiable medium. The identification apparatus may be a barcode reader, or a two-dimensional code reader, or a three-dimensional code reader, or an NFC reader, or an RFID reader, and reference may be made to the foregoing embodiments for details, which will not be repeated here. Moreover, the scanning probe 701 performs, according to the scan-related information, a secondary scan on the subject to be scanned. For example, the processor sets, according to the scan parameter information, a scan parameter of the secondary scan, and/or, the position and pose of the scanning probe 701 when performing the secondary scan are determined according to the body marker information.

In some embodiments, the processor 702 obtains an updated medical image and updated scan-related information on the basis of adjusted information of at least one of the scan parameter information and the body marker information, and the processor 702 stores the updated scan-related information in the identifiable medium.

In some embodiments, the medical device further includes: a display 704 that superimposes and displays the scan-related information on a medical image obtained by the secondary scan. The processor 702 generates a body marker image according to the body marker information of the scan-related information, the body marker image including a visual representation image of a scanned site and a scanning probe icon superimposed and displayed on the visual representation image, and the display 704 superimposes and displays the body marker image on the medical image obtained by the secondary scan.

FIG. 8 is a schematic diagram of an ultrasound device according to an embodiment of the present application. As shown in FIG. 8, the ultrasound device 800 may be configured to provide ultrasound imaging, and may therefore include suitable circuitry, interfaces, logic, and/or code for performing and/or supporting ultrasound imaging-related functions.

The ultrasound device includes, for example, a transmitter 202, an ultrasound probe 204 (scanning probe), a transmitting beamformer 210, a receiver 218, a receiving beamformer 220, an RF processor 224, an RF/IQ buffer 226, a user input module 230, a signal processor 240 (processor), an image buffer 250, a display system 260 (display), a file 270, and an identification apparatus 280.

The transmitter 202 may include suitable circuitry, interfaces, logic, and/or code operable to drive the ultrasound probe 204. The ultrasound probe 204 may include an array of two-dimensional (2D) piezoelectric elements. The ultrasound probe 204 may include a set of transmitting transducer elements 206 and a set of receiving transducer elements 208 that typically form the same element. In some embodiments, the ultrasound probe 204 may be operable to acquire ultrasound image data covering at least a substantial portion of an anatomical structure.

The transmitting beamformer 210 may include suitable circuitry, interfaces, logic, and/or code that is operable to control the transmitter 202, and the transmitter 202 drives the set of transmitting transducer elements 206 through a transmitting subaperture beamformer 214 to transmit ultrasound emission signals into a region of interest (e.g., a person, animal, subsurface cavity, physical structure, etc.). The emitted ultrasound signal can be backscattered from structures in the subject of interest (e.g., blood cells or tissue) to produce echoes. The echo is received by the receiving transducer element 208.

The set of receiving transducer elements 208 in the ultrasonic probe 204 may be operated to convert the received echo to an analog signal for subaperture beam formation through a receiving subaperture beamformer 216, which is then transmitted to the receiver 218. The receiver 218 may include suitable circuitry, interfaces, logic, and/or code that is operable to receive signals from the receiving subaperture beamformer 216. The analog signal can be transferred to one or more of a plurality of A/D converters 222.

The plurality of A/D converters 222 may include suitable circuitry, interfaces, logic, and/or code that is operable to convert the analog signal from the receiver 218 to a corresponding digital signal. The plurality of A/D converters 222 are provided between the receiver 218 and the RF processor 224. Nevertheless, the present disclosure is not limited in this regard. Thus, in some embodiments, the plurality of A/D converters 222 may be integrated within the receiver 218.

The RF processor 224 may include suitable circuitry, interfaces, logic, and/or code that is operable to demodulate the digital signals output by the plurality of A/D converters 222. According to one embodiment, the RF processor 224 may include a complex demodulator (not shown) that is operable to demodulate the digital signal to form an I/Q data pair representing the corresponding echo signal. The RF or I/Q signal data can then be transferred to the RF/IQ buffer 226. The RF/IQ buffer 226 may include suitable circuitry, interfaces, logic, and/or code that is operable to provide temporary storage of RF or I/Q signal data generated by the RF processor 224.

The receiving beamformer 220 may include suitable circuitry, interfaces, logic, and/or code that may be operable to perform digital beamforming processing to, for example, sum and output a beam summing signal for the delay-channel signals received from the RF processor 224 via the RF/IQ buffer 226. The resulting processed information may be the beam summing signal output from the receiving beamformer 220 and transmitted to the signal processor 240. According to some embodiments, the receiver 218, the plurality of A/D converters 222, the RF processor 224, and the beamformer 220 may be integrated into a single beamformer which may be digital. In various embodiments, the ultrasound system 200 includes a plurality of receiving beamformers 220.

The user input device 230 can be used to enter patient data, scan parameters, and settings, and select protocols and/or templates to interact with the AI segmentation processor, so as to select tracking targets, etc. In an illustrative embodiment, the user input device 230 is operable to configure, manage, and/or control the operation of one or more components and/or modules in the ultrasound system 200. In this regard, the user input device 230 is operatable to configure, manage, and/or control the operation of the transmitter 202, the ultrasound probe 204, the transmitting beamformer 210, the receiver 218, the receiving beamformer 220, the RF processor 224, the RF/IQ buffer 226, the user input device 230, the signal processor 240, the image buffer 250, the display system 260, and/or the file 270.

For example, the user input device 230 may include a button, a knob, a touch screen, motion tracking, voice recognition, a mouse device, a keyboard, a trackball, a camera, and/or any other devices capable of receiving user instructions. In some embodiments, for example, one or more of the user input devices 230 may be integrated into other components (such as the display system 260 or the ultrasound probe 204). As an example, the user input device 230 may include a touch screen display. As another example, the user input device 230 may determine the angle of rotation of the ultrasound probe according to the body marker image, and adjust the pose of the ultrasound probe by rotating the knob to the angle of rotation.

The signal processor 240 may include suitable circuitry, interfaces, logic, and/or code that is operable to process the ultrasound scan data (i.e., the summed IQ signal) to generate an ultrasound image for presentation on the display system 260. The signal processor 240 is operable to perform one or more processing operations based on a plurality of selectable ultrasound modalities on the acquired ultrasound scan data. In an illustrative embodiment, the signal processor 240 is operable to perform display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various embodiments, the processed image data may be presented at the display system 260 and/or may be stored in the file 270. The file 270 can be a local file, a picture archiving and communication system (PACS), or any suitable device for storing images and related information.

The signal processor 240 may be one or more central processing units, microprocessors, microcontrollers, etc. For example, the signal processor 240 may be an integrated component, or may be distributed in various locations. The signal processor 240 may be configured to receive input information from the user input device 230 and/or file 270, generate outputs that may be shown by the display system 260, and manipulate the outputs, etc., in response to the input information from the user input device 230. The signal processor 240 may be capable of executing, for example, any of one or more of the methods and/or one or more sets of instructions discussed herein according to various embodiments.

The ultrasound device 800 is operable to continuously acquire ultrasound scan data at a frame rate suitable for the imaging situation under consideration. Typical frame rates are in the range of 20 to 220, but can be lower or higher. The acquired ultrasound scan data can be shown on the display system 260 in real-time at a display rate that is the same as the frame rate, or slower, or faster than the frame rate. The image buffer 250 is included to store frames for processing of the acquired ultrasound scan data that are not scheduled for immediate display. Preferably, the image buffer 250 has sufficient capacity to store frames of ultrasound scan data for at least a few minutes. Frames of ultrasound scan data are stored in such a way that they can be easily retrieved therefrom according to their acquisition sequence or time. The image buffer 250 may be embodied in any known data storage medium.

In some specific embodiments, the signal processor 240 may be configured to perform or otherwise control at least some of the functions performed thereby based on user instructions via the user input device 230. As an example, the user may provide voice commands, probe poses, button presses, etc. to issue specific commands such as controlling aspects of automatic strain measurement and strain ratio calculations, and/or provide or otherwise specify various parameters or settings associated therewith, as described in more detail below.

In operation, the ultrasound device 800 may be used to generate an ultrasound image, including a two-dimensional (2D) image, a three-dimensional (3D) image, and/or a four-dimensional (4D) image. In this regard, the ultrasound system 200 may be operated to continuously acquire ultrasound scan data at a specific frame rate, which may be applicable to the imaging situation discussed. For example, the frame rate can be in the range of 20-70, or can be lower or higher. The acquired ultrasound scan data can be shown on the display system 260 at the same display rate as the frame rate, or slower, or faster than the frame rate. The image buffer 250 is included to store frames for processing of the acquired ultrasound scan data that are not scheduled for immediate display. Preferably, the image buffer 250 has sufficient capacity to store at least a few seconds of frames of ultrasound scan data. Frames of ultrasound scan data are stored in such a way that they can be easily retrieved therefrom according to their acquisition sequence or time. The image buffer 250 may be embodied in any known data storage medium.

In some embodiments, the ultrasound probe 204 scans a subject to be scanned, and the signal processor 240 obtains a medical image obtained by a current scan, and current scan-related information, and stores the scan-related information in an identifiable medium, the identifiable medium being provided in a portable mobile carrier.

In some embodiments, the identification apparatus 280 identifies the scan-related information in the identifiable medium. The identification apparatus may be a barcode reader, or a two-dimensional code reader, or a three-dimensional code reader, or an NFC reader, or an RFID reader, and reference may be made to the foregoing embodiments for details, which will not be repeated here. Moreover, the ultrasound probe 204 performs, according to the scan-related information, a secondary scan on the subject to be scanned. For example, the signal processor 240 sets, according to the scan parameter information, a scan parameter of the secondary scan, and/or, the position and pose of the ultrasound probe 204 when performing the secondary scan is determined according to the body marker information. For example, the user input device 230 may determine the angle of rotation of the ultrasound probe according to the body marker image, and adjust the pose of the ultrasound probe by rotating the knob to the angle of rotation.

In some embodiments, the signal processor 240 obtains an updated medical image and updated scan-related information on the basis of adjusted information of at least one of the scan parameter information and the body marker information, and the signal processor 240 stores the updated scan-related information in the identifiable medium.

In some embodiments, the display system 260 superimposes and displays the scan-related information on a medical image obtained by the secondary scan. The signal processor 240 generates a body marker image according to the body marker information of the scan-related information, the body marker image including a visual representation image of a scanned site and a scanning probe icon superimposed and displayed on the visual representation image, and the display system 260 superimposes and displays the body marker image on the medical image obtained by the secondary scan.

The embodiments of the present application further provide a computer-readable program, wherein the program, when executed, causes a computer to perform, in a medical device, the medical scanning method described in the foregoing embodiments.

The embodiments of the present application further provide a storage medium storing a computer-readable program, wherein the computer-readable program causes a computer to perform, in a medical device, the medical scanning method described in the foregoing embodiments.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A medical device, comprising:
a scanning probe, the scanning probe configured to scan a subject;
a memory storing instructions' and
a processor configured to execute the instructions to:
obtain a medical image and scan-related information obtained by scanning the subject with the scanning probe; and
store the scan-related information in an identifiable medium, the identifiable medium being provided in a portable mobile carrier.

2. The medical device according to claim 1, wherein the identifiable medium comprises at least one of a barcode, a two-dimensional code, a three-dimensional code, an NFC tag, and an RFID tag.

3. The medical device according to claim 1, wherein the portable mobile carrier comprises at least one of an electronic mobile terminal, a printable medium, and an integrated circuit card.

4. The medical device according to claim 1, wherein the scan-related information comprises at least one of scan parameter information, body marker information, and scanning result information.

5. The medical device according to claim 4, wherein the processor is further configured to:
identify the scan-related information in the identifiable medium; and
control the scanning probe to perform a secondary scan on the subject based on the scan-related information.

6. The medical device according to claim 5, wherein the processor is further configured to execute the instructions to set a scan parameter of the secondary scan based on the scan parameter information.

7. The medical device according to claim 5, wherein the position and the pose of the scanning probe when performing the secondary scan are determined according to the body marker information.

8. The medical device according to claim 5, wherein the processor is further configured to execute the instructions to:
obtain an updated medical image and updated scan-related information based on adjusted information of at least one of the scan parameter information and the body marker information; and
store the updated scan-related information in the identifiable medium.

9. The medical device according to claim 6, further comprising: a display;
wherein the processor is further configured to execute the instructions to:
generate a body marker image according to the body marker information of the scan-related information, the body marker image comprising a visual representation image of a scanned site and a scanning probe icon superimposed and displayed on the visual representation image; and
control the display to display a superimposed body marker image on a medical image obtained by the secondary scan.

10. A medical scanning method, comprising:
scanning a subject with a medical scanning device;
obtaining a medical image and scan-related information based on the scanning; and
storing the scan-related information in an identifiable medium, the identifiable medium being provided in a portable mobile carrier.

11. The scanning method according to claim 1, wherein the identifiable medium comprises at least one of a barcode, a two-dimensional code, a three-dimensional code, an NFC tag, and an RFID tag.

12. The scanning method according to claim 1, wherein the portable mobile carrier comprises at least one of an electronic mobile terminal, a printable medium, and an integrated circuit card.

13. The scanning method according to claim 1, wherein the scan-related information comprises at least one of scan parameter information, body marker information, and scanning result information.

14. The scanning method according to claim 13, wherein the scan parameter information comprises at least one of a scanning mode, a scanning presetting, a scan parameter adjustment, and a scanning time;
the body marker information comprises body marker type information and position and pose information of a scanning probe icon; the position and pose information comprises at least one of position information of the scanning probe icon and angle of rotation information of the scanning probe icon; and
the scanning result information comprises at least one of a measurement result and diagnostic information.

15. The scanning method according to claim 14, wherein the position and pose information is information of the position of the scanning probe icon and/or the angle of rotation of the scanning probe icon in a visual representation image coordinate system of a scanned site.

16. The scanning method according to claim 13, further comprising:
identifying the scan-related information in the identifiable medium; and
performing a secondary scan on the subject based on the scan-related information.

17. The scanning method according to claim 16, wherein the performing the secondary scan based on the scan-related information comprises at least one of the following:
setting, according to the scan parameter information, a scan parameter of the secondary scan; and
determining, according to the body marker information, the position and the pose of a scanning probe used in the secondary scan.

18. The scanning method according to claim 16, further comprising:
adjusting at least one of the scan parameter information and the body marker information to obtain an updated medical image and updated scan-related information; and
storing the updated scan-related information in the identifiable medium.

19. The scanning method according to claim 16, further comprising:
superimposing and displaying the scan-related information on a medical image obtained by the secondary scan.

20. The scanning method according to claim 19, further comprising:
generating a body marker image according to the body marker information of the scan-related information, the body marker image comprising a visual representation image of a scanned site and a scanning probe icon superimposed and displayed on the visual representation image; and
superimposing and displaying the body marker image on the medical image obtained by the secondary scan.
